SEQUENCE LISTING (15 sequences)

SEQ ID NO: 1 - DNA, 20 bp, Artificial Sequence
  VEGFR2  1191 C/T Forward Primer
tcttggtcatcagcccactg

SEQ ID NO: 2 - DNA, 20 bp, Artificial Sequence
  VEGFR2 1191 C/T Reverse Primer
aaacccagtctgggagtgag

SEQ ID NO: 3 - DNA, 24 bp, Artificial Sequence
  FLT2 738 T/C Forward Primer
gcagctgtaaagaagaaagtccag

SEQ ID NO: 4 - DNA, 18 bp, Artificial Sequence
  FLT3 738 T/C/ Reverse Primer
tgcattccctgcccagtt

SEQ ID NO: 5 - DNA, 20 bp, Artificial Sequence
  ABCB1 1236 T/C Forward Primer
tgtctgtgaattgccttgaa

SEQ ID NO: 6 - DNA, 20 bp, Artificial Sequence
  ABCB1 1236 T/C Reverse Primer
ggtcatagagcctctgcatc

SEQ ID NO: 7 - DNA, 21 bp, Artificial Sequence
  ABCB1 2677 G/TA Forward Primer
gtacccatcattgcaatagca

SEQ ID NO: 8 - DNA, 23 bp, Artificial Sequence
  ABCB1 2677 G/TA Reverse Primer
tttagtttgactcaccttcccag

SEQ ID NO: 9 - DNA, 21 bp, Artificial Sequence
  ABCB1 3435 C/T Forward Primer
gagcccatcctgtttgactgc

SEQ ID NO: 10 - DNA, 25 bp, Artificial Sequence
  ABCB1 3435 C/T Reverse Primer
cattaggcagtgactcgatgaaggc

SEQ ID NO: 11 - DNA, 23 bp, Artificial Sequence
  ABCG2 421 C/A Forward Primer
ggatgatgttgtgatgggcactc

SEQ ID NO: 12 - DNA, 26 bp, Artificial Sequence
  ABCG2 421 C/A  Reverse Primer
ccagacctaactcttgaatgaccctg

SEQ ID NO: 13 - DNA, 21 bp, Artificial Sequence
  BIM i2dela Forward Primer (1)
cttagcgtaatgtcgtcaggg

SEQ ID NO: 14 - DNA, 22 bp, Artificial Sequence
  BIM i2dela Forward Primer (2)
aggcttcagtgaggtaaatcac

SEQ ID NO: 15 - DNA, 22 bp, Artificial Sequence
  BIM i2dela  Reverse Primer
gctcctctgtgaggccagcctg

(19) United States
(12) Patent Application Publication
(10) Pub. No.: US 2016/0251725 A1
(43) Pub. Date: Sep. 1, 2016

(54) METHOD FOR PREDICTING CLINICAL TOXICITY AND OUTCOME

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Min-Han TAN, Singapore (SG); Ying Hsia CHU, Singapore (SG); Johnathan LAI, Singapore (SG); Puay Hoon TAN, Singapore (SG)

(21) Appl. No.: 15/026,957

(22) PCT Filed: Oct. 3, 2014
(86) PCT No.: PCT/SG2014/000467

§ 371 Date: Apr. 1, 2016

(30) Foreign Application Priority Data
Oct. 3, 2013 (SG) 201307439-8

(51) Int. Cl. C12Q 1/68

(52) U.S. Cl. CPC C12Q 1/6886; C12Q 2600/158; C12Q 2600/156; C12Q 2600/106

(57) ABSTRACT

A method for determining the sensitivity of a patient with cancer to receptor tyrosine kinase inhibitor therapy is provided. The method comprising screening a nucleic acid sample to determine the identity of at least one single nucleotide polymorphism (SNP) genotype selected from the group consisting of rs1933437, rs1045642, rs1128503, rs2032582 and rs2231142 and any combination thereof; and determining the sensitivity of a patient with renal cancer to receptor tyrosine kinase inhibitor therapy based on the identity of the SNP genotype. The sensitivity is selected from one or more of neutropenia, diarrhea, tumor response, early toxicity-necessitated treatment termination, overall survival and progression-free survival.

### FIELD OF THE INVENTION

The present invention relates stem cells. In particular the present invention relates to a method for culturing and maintaining a pluripotent stem cell in an undifferentiated state.

### BACKGROUND OF THE INVENTION

Cancer therapy includes surgery, chemotherapy, targeted therapy, immunotherapy and radiation therapy. Targeted therapy is directed against cancer-specific molecules and signaling pathways within the tumor. Accordingly, drugs used in targeted therapy include drugs that inhibit angiogenesis and drugs that induce apoptosis. An example of a class of drugs used in targeted therapy is tyrosine kinase inhibitors (TKIs).

However, tumor response and individual survival vary among patients and therefore the therapeutic benefits of TKIs are not universal but individualized. In addition, despite its effectiveness in prolonging survival, life quality-compromising and potentially lethal toxicities are common. These include diarrhea, hand-foot syndrome, mucositis, hypertension, leucopenia, neutropenia and thrombocytopenia. Toxicities lead to dose interruption, dose reduction and prevent patients from benefiting fully from the drug. Toxicity presentation and severity, like survival, are also not universal but vary greatly among patients.

Therefore, there is a need to provide a method for determining the sensitivity of a patient with cancer to TKI therapy and a need to provide a method of identifying the most appropriate TKI therapy regimen for a patient. There is also a need for a kit to be used in such methods.

### SUMMARY OF THE INVENTION

In a first aspect, there is provided a method for determining the sensitivity of a patient with cancer to receptor tyrosine kinase inhibitor therapy, comprising:
a) isolating a nucleic acid sample from a biological sample obtained from the said patient;
b) screening said nucleic acid sample to determine the identity of at least one single nucleotide polymorphism (SNP) genotype selected from the group consisting of rs1933437, rs1045642, rs1128503, rs2032582 and rs2231142 and any combination thereof; and
c) determining the sensitivity of a patient with renal cancer to receptor tyrosine kinase inhibitor therapy based on the identity of at least one SNP genotype selected from the group consisting of rs1933437, rs1045642, rs1128503, rs2032582 and rs2231142 and any combination thereof,
wherein said sensitivity is selected from one or more of neutropenia, diarrhea, tumor response, early toxicity-necessitated treatment termination, overall survival and progression-free survival.

In a second aspect, there is provided a use of the method as described herein to identify the most appropriate receptor tyrosine kinase inhibitor therapy regimen for a patient with renal cancer.

In a third aspect, there is provided a kit for use according to the method as described herein, comprising components for the screening of said nucleic acid sample to determine the identity of at least one SNP genotype selected from the group consisting of rs1933437, rs1045642, rs1128503, rs2032582 and rs2231142 and any combination thereof.

### DEFINITIONS

The following words and terms used herein shall have the meaning indicated:

The term subject refers to living, multicellular vertebrate organisms, a category that includes both human and veterinary subjects for example, mammals, birds and primates.

The term nucleic acid refers to a deoxyribonucleotide or ribonucleotide polymer in either single or double stranded form, and unless otherwise limited, encompassing known analogues of natural nucleotides that hybridize to nucleic acids in a manner similar to naturally occurring nucleotides.

The term polymorphism refers to a variant in a sequence of a gene, usually carried from one generation to another in a population. Polymorphisms can be those variations (nucleotide sequence differences) that, while having a different nucleotide sequence, produce functionally equivalent gene products, such as those variations generally found between individuals, different ethnic groups, geographic locations. The term polymorphism also encompasses variations that produce gene products with altered function, i.e., variants in the gene sequence that lead to gene products that are not functionally equivalent. This term also encompasses variations that produce no gene product, an inactive gene product, or increased or increased activity gene product.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation (e.g., an alteration of a secondary structure such as a stem-loop, or an alteration of the binding affinity of the nucleic acid for associated molecules, such as polymerases, RNases, and so forth).

The term Single Nucleotide Polymorphism (SNP) refers to a single base (nucleotide) difference in a specific location in the DNA sequence among individuals in a population. A subset of SNPs give rise to changes in the encoded amino acid sequence; these are referred to as coding SNPs, or cSNPs.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF THE DRAWINGS

The invention will be better understood with reference to the detailed description when considered in conjunction with the non-limiting examples and the accompanying drawings, in which:

FIG. 1 shows (A) Patients grouped according to the ABCB1 3435C/T, 1236C/T, 2677G/T haplotype; median progression-free survival for homozygous carriers of the TTT haplotype was 2.4 months and for other cases was 8.4 months (P=0.001). (B) Patients grouped according to the ABCB1 3435C/T, 1236C/T, 2677G/TA haplotype; median overall survival for homozygous carriers of the TTT haplotype was 4.6 months and for other cases was 19.6 months (P=0.005).

### DETAILED DESCRIPTION OF THE PRESENT INVENTION

In a first aspect there is provided a method for determining the sensitivity of a patient with cancer to receptor tyrosine kinase inhibitor therapy. The method may comprise: isolating a nucleic acid sample from a biological sample obtained from the said patient; screening said nucleic acid sample to determine the identity of at least one single nucleotide polymorphism (SNP) genotype selected from the group consisting of rs1933437, rs1045642, rs1128503, rs2032582 and rs2231142 and any combination thereof; and determining the sensitivity of a patient with renal cancer to receptor tyrosine kinase inhibitor therapy based on the identity of at least one SNP genotype selected from the group consisting of rs1933437, rs1045642, rs112, 8503, rs2032582 and rs2231142 and any combination thereof, wherein said sensitivity is selected from one or more of neutropenia, diarrhea, tumor response, early toxicity-necessitated treatment termination, overall survival and progression-free survival.

In one embodiment, the cancer may be selected from the group consisting of renal cancer, neuroendocrine cancer, breast cancer, prostate cancer, cervical cancer, ovarian cancer, gastric cancer, colorectal cancer, pancreatic cancer, liver cancer, brain cancer, lung cancer, hematological malignancies, melanoma and sarcomas.

In another embodiment, the renal cancer is metastatic renal cell carcinoma (mRCC). In some embodiments the patient may suffer from mRCC. Alternatively, the patient suffers from mRCC and undergoes receptor tyrosine kinase inhibitor treatment.

In some embodiments, the biological sample is obtained from the group consisting of frozen tissue, tissue biopsies, circulating cells, bodily fluids and cheek swab.

The biological sample may be non-neoplastic or neoplastic.

In one embodiment the bodily fluids may be selected from the group consisting of blood, saliva, ascites, effusions and urine.

The receptor tyrosine kinase inhibitor may be a multikinase inhibitor for receptor tyrosine kinase including but not limited to sunitinib, pazopanib, axitinib, sorafenib, regorafenib, tivozantinib or combinations thereof.

In some embodiments the patient is a mammal or a human. The human may be an ethnic Asian, or an ethnic Caucasian, or an ethnic African.

In another embodiment, for determining the risk of diarrhea during receptor tyrosine kinase inhibitor treatment, the method may further comprise: (a) screening for at least one SNP genotype selected from the group consisting of rs1128503 and rs1045642, and (b) determining the risk of diarrhea based on the screening result obtained in step (a). In some embodiments, in step (b), with respect to rs1128503, the risk of diarrhea during receptor tyrosine kinase inhibitor treatment may be higher for a CC genotype than a CT or TT genotype. In some embodiments, in step (b), with respect to rs1045642, the risk of diarrhea during receptor tyrosine kinase inhibitor treatment may be higher for a CC genotype than a CT or TT genotype.

In some embodiments, for determining the risk of neutropenia during receptor tyrosine kinase inhibitor treatment, the method may further comprise: (a) screening for at least one SNP genotype selected from the group consisting of rs1933437, rs2032582, rs1045642, rs1128503 and rs2231142, and (b) determining the risk of neutropenia based on the screening result obtained in step (a). In step (b), with respect to rs1933437, the risk of neutropenia during receptor tyrosine kinase inhibitor treatment may be higher for a TT genotype than a CC or CT genotype. In step (b), with respect to rs2032582, the risk of neutropenia during receptor tyrosine kinase inhibitor treatment may be higher for a AA or AG or GG genotype than a AT or GT or TT genotype. In step (b), with respect to rs1045642, the risk of neutropenia during receptor tyrosine kinase inhibitor treatment may be higher for a CC or CT genotype than a TT genotype. In step (b), with respect to rs1128503, the risk of neutropenia during receptor tyrosine kinase inhibitor treatment may be higher for a CC or CT genotype than a TT genotype. In step (b), with respect to rs2231142, the risk of neutropenia during receptor tyrosine, kinase inhibitor treatment may be higher for a CC or AC genotype than a AA genotype. In step (b), with respect to the three SNPs including rs1045642, rs2032582 and rs1128503, the risk of neutropenia during receptor tyrosine kinase inhibitor treatment may be higher for allele combinations other than T allele homozygosity at all of the three SNP sites than for a TTT/TTT genotype.

In some embodiments, for determining the risk of early toxicity-necessitated treatment termination during receptor tyrosine kinase inhibitor treatment, the method may further comprise: (a) screening for the SNP genotype of rs1128503, and (b) determining the risk of early toxicity-necessitated treatment termination based on the genotyping result obtained in step (a).

In step (b), with respect to rs1128503, the risk of early toxicity-necessitated treatment termination during receptor tyrosine kinase inhibitor treatment may be higher for a CC or CT genotype than a TT genotype.

In some embodiments, for determining the risk of primary tumour resistance to receptor tyrosine kinase inhibitor treatment, the method may further comprise: (a) screening for at least one SNP genotype selected from the group consisting of rs1045642, rs2032582 and rs1128503, and (b) determining the risk of primary tumour resistance to receptor tyrosine kinase inhibitor treatment based on the genotyping result obtained in step (a).

In step (b), with respect to rs1045642, the risk of primary tumour resistance to receptor tyrosine kinase inhibitor treatment may be higher for a TT genotype than a CC or CT genotype.

In step (b), with respect to rs2032582, the risk of primary tumour resistance to receptor tyrosine kinase inhibitor treatment may be higher for a TT genotype than a AA or AG or GG or AT or GT genotype.

In step (b), with respect to the three SNPs including rs1045642, rs2032582 and rs1128503, the risk of primary tumour resistance to receptor tyrosine kinase inhibitor therapy may be higher for a TTT/TTT genotype than for allele combinations other than T allele homozygosity at all of the three SNP sites.

In some embodiments, for determining overall survival, during receptor tyrosine kinase inhibitor treatment, the method may further comprise: (a) screening for at least one SNP genotype selected from the group consisting of rs1045642, rs2032582 and rs1128503, and (b) determining overall survival, based on the genotyping result obtained in step (a).

In step (b), with respect to the three SNPs including rs1045642, rs2032582 and rs1128503, overall survival during receptor tyrosine kinase inhibitor treatment, may be lower for a TTT/TTT genotype than for allele combinations other than T allele homozygosity at all of the three SNPs.

In some embodiments, for determining progression-free survival, during receptor tyrosine kinase inhibitor treatment, the method may further comprise: (a) screening for at least one SNP genotype selected from the group consisting of rs1045642, rs2032582 and rs1128503, and (b) determining progression-free survival, based on the genotyping result obtained in step (a).

In step (b), with respect to the three SNP genotypes rs1045642, rs2032582 and rs1128503, progression-free survival during receptor tyrosine kinase inhibitor treatment may be lower for a TTT/TTT genotype than for allele combinations other than T allele homozygosity at all of the three SNPs.

In another aspect, there is provided the use of the method as described herein to identify the most appropriate receptor tyrosine kinase inhibitor therapy regimen for a patient with renal cancer.

In another aspect, there is provided a kit for use in accordance with the method as described herein, comprising components for the screening of said nucleic acid sample to determine the identity of at least one SNP genotype selected from the group consisting of rs1933437, rs1045642, rs1128503, rs2032582 and rs2231142 and any combination thereof.

### EXPERIMENTAL SECTION

### Example 1

### Evaluation of Genetic Markers for Determining Toxicity, Response and Survival in Patients with Cancer

Materials and Methods

Patients and Treatment

A total of 97 mRCC patients receiving sunitinib between 2006 and 2014 at National Cancer Centre Singapore (NCCS) were included in this retrospective study, which was approved by the Institutional Review Board (Singapore Health Services). The majority of patients (79/97) received sunitinib at a starting dose of 37.5 mg daily over 4 consecutive weeks followed by a 2 week break. This attenuation from the drug label-recommended dosage of 50 mg daily was established as routine at NCCS after severe to life-threatening toxicities were frequently noted in early use of sunitinib when initiated at 50 mg daily. Twelve patients in this study received a starting dose of 50 mg daily. Six patients started with 25 mg daily due to advanced age or patients' aversion to expected toxicities.

Follow-Up and Data Collection

Sunitinib toxicities and tumor response were evaluated by independent physicians in every treatment cycle based on Common Terminology Criteria for Adverse Events (CTCAE) version 3.0 and Response Evaluation Criteria in Solid Tumors (RECIST) criteria version 1.1. Laboratory assessments of serum creatinine, total bilirubin, albumin, aspartate transaminase, alanine transaminase, hemoglobin, leucocytes and platelets, and clinical examinations for mucositis, hand-foot syndrome, fatigue and diarrhea were conducted at baseline (before starting sunitinib), and at two time points in each cycle: after 4 weeks of daily sunitinib, and after 2 weeks of sunitinib-free rest (before starting the next cycle). Patient characteristics were also collected including age, gender, ethnicity, body weight and height, and Eastern Cooperative Oncology Group (ECOG) performance status. Memorial Sloan-Kettering Cancer Center (MSKCC) prognostic score was calculated for each patient with available data. All the collected data were de-identified by a third party before being used in statistical analysis. The follow-up period ended at the end of April, 2014.

Toxicity Definitions

The toxicities analyzed included leucopenia, neutropenia, thrombocytopenia, diarrhea, hand-foot syndrome and early toxicity-necessitated treatment termination. Blood cell counts from the electronic medical system and the physician notes from the first sunitinib cycle were assessed for leucopenia (<3000/μL), neutropenia (<2000/μL), thrombocytopenia (<150000/μL), hand-foot syndrome (documented physical examination finding) and diarrhea (documented patient complaints). Early toxicity-necessitated treatment termination was defined as interruption of the first or second treatment cycle consented by the patient and the physician based on recognition of intolerable or life-threatening toxicities without evidence of progressive disease and failure to resume treatment as far as the patient was followed. For patients experiencing any toxicity grade≧2, cycle interruptions were frequently required; in such cases, a resumed treatment with 25% dose reduction would be applied for safety and life quality considerations.

Survival Endpoint Definition

Progression free survival was defined as the time from the date of suitinib initiation to the date of sunitinib termination if suitinib was terminated due to radiological or clinical evidence of progressive disease, toxicities or death. Overall survival was defined as the time from the date of sunitinib initiation to the date of death, or to the date of the last follow up for censored cases.

Genotyping

6 SNPs were genotyped, as listed in Table 1, in 4 genes: sunitinib targets FLT3 and VEGFR2, and multi-drug effluxers ABCG2 and ABCB1. These polymorphisms were selected based on minor allele frequency higher than 0.1 in Han Chinese, previously reported associations with sunitinib toxicities (Table 2), and presumed function in sunitinib pharmacokinetics or pharmacodynamics. Primers for genotyping the SNPs and the BIM deletion are provided in Table 3.

TABLE 1
Polymorphisms genotyped and allele frequencies

| | | | | Genotype | | | |
|---|---|---|---|---|---|---|---|
| | | | | distribution | | | |
| | | | | wt/ | wt/ | var/ | |
| Polymorphism | rs number | Variation | n^{a} | wt | var | var | VAF^{d} |
| VEGFR2 | rs2305948 | V297I | 94 | 64 | 30 | 0 | 0.160 |
| 1191 C/T | | | | | | | (T) |
| FLT3 738 | rs1933437 | M227T | 95 | 47 | 43 | 5 | 0.279 |
| T/C | | | | | | | (C) |
| ABCB1 | rs1128503 | G412G | 93 | 35 | 49 | 9 | 0.360 |
| 1236 T/C | | | | | | | (C) |
| ABCB1 | rs2032582 | A893S/T | 96 | 25 | 44^{b} | 27^{c} | 0.375 |
| 2677 G/TA | | | | | | | (T); |
| | | | | | | | 0.135 |
| | | | | | | | (A) |
| ABCB1 | rs1045642 | I1145I | 96 | 34 | 50 | 12 | 0.385 |
| 3435 C/T | | | | | | | (T) |
| ABCG2 421 | rs2231142 | Q141K | 95 | 50 | 38 | 7 | 0.274 |
| C/A | | | | | | | (A) |

^{a}Patients successfully genotyped.
^{b}include 34 GT and 10 AG individuals.
^{c}include 2 AA, 12 AT and 13 TT individuals.
^{d}Variant allele frequencies.

TABLE 2
Previous analyses on the 6 SNPs selected for this
study.

| | Significantly | | |
|---|---|---|---|
| | associated | | |
| SNP | outcomes | Cohort size | Publication |
| VEGFR2 1191 | Any toxicity | (n = 183) | van Erp et |
| C/T | grade>2 | | al., 2009 |
| | increased with T | | |
| | allele. | | |
| FLT3 738 T/C | Leucopenia | (n = 188) | van Erp et |
| | increased with T | | al., 2009 |
| | allele. | | |
| ABCB1 1236 T/C | Poorer PFS and | (n = 88) | Beuselinck et |
| | OS with the TT | | al., 2013 |
| | genotype. | | |
| | Less dose | (n = 96) | Beuselinck et |
| | reductions with | | al., 2014 |
| | the TT genotype. | | |
| ABCB1 2677G/TA | Less dose | (n = 96) | Beuselinck et |
| | reductions with | | al., 2014 |
| | TT or TA | | |
| | genotypes. | | |
| ABCB1 | HFS increased | (n = 219) | van Erp et |
| haplotype^{a} | with TTT | | al., 2009 |
| | haplotype. | | |
| | PFS increased | (n = 136) | van der Veldt |
| | with TCG | | et al., 2011 |
| | haplotype. | | |
| ABCG2 421 C/A | More | (n = 65) | Kim et al., |
| | thrombocytopenia, | | 2013 |
| | neutropenia, | | |
| | and HFS with the | | |
| | AA genotype. | | |

Abbreviations:
OS, overall survival;
PFS, progression-free survival;
HFS, hand-foot syndrome.
^{a}ABCB1 haplotype composed of 3435 C/T, 1236 T/C and 2677 G/TA.

TABLE 3
Primers for genotyping

| Gene | Polymorphism | rs Number | Forward Primer | Reverse Primer |
|---|---|---|---|---|
| VEGFR2 | 1191 C/T | rs2305948 | 5′TCTTGGTCATC | 5′AAACCCAGTCTGG |
| | | | AGCCCACTG3′ | GAGTGAG3′ |
| | | | (SEQ ID NO. 1) | (SEQ ID NO. 2) |
| FLT3 | 738 T/C | rs1933437 | 5′GCAGCTGTAAA | 5′TGCATTCCCTGCC |
| | | | GAAGAAAGTCCAG3′ | CAGTT3′ |
| | | | (SEQ ID NO. 3) | (SEQ ID NO. 4) |
| ABCB1 | 1236 T/C | rs1128503 | 5′TGTCTGTGAAT | 5′GGTCATAGAGCCT |
| | | | TGCCTTGAA3′ | CTGCATC3′ |
| | | | (SEQ ID NO. 5) | (SEQ ID NO. 6) |
| ABCB1 | 2677 G/TA | rs2032582 | 5′GTACCCATCAT | 5′TTTAGTTTGACTC |
| | | | TGCAATAGCA3′ | ACCTTCCCAG3′ |
| | | | (SEQ ID NO. 7) | (SEQ ID NO. 8) |
| ABCB1 | 3435 C/T | rs1045642 | 5′GAGCCCATCCT | 5′CATTAGGCAGTGA |
| | | | GTTTGACTGC3′ | CTCGATGAAGGC3′ |
| | | | (SEQ ID NO. 9) | (SEQ ID NO. 10) |
| ABCG2 | 421 C/A | rs2231142 | 5′GGATGATGTTG | 5′CCAGACCTAACTC |
| | | | TGATGGGCACTC3′ | TTGAATGACCCTG3′ |
| | | | (SEQ ID NO. 11) | (SEQ ID NO. 12) |
| BIM | BIM i2del^{a} | — | (1)5′CTTAGCGT | 5′GCTCCTCTGTGAG |
| | | | AATGTCGTCAGGG3′ | GCCAGCCTG3′ |
| | | | (SEQ ID NO. 13) | (SEQ ID NO. 15) |
| | | | (2)5′AGGCTTCA | |
| | | | GTGAGGTAAATCAC3′ | |
| | | | (SEQ ID NO. 14) | |

^{a}a 2,903-bp deletion polymorphism in intron 2 of BIM associated with resistance to tyrosine kinase inhibitors.

Germline DNA was obtained from peripheral blood, formalin-fixed tissue of benign kidney from nephrectomy and immortalized lymphocytes. The labeling on blood tubes and tissue slides were de-identified by a third party before being used for DNA extraction or cell immortalization. Genotyping was done by PCR amplification of the flanking region of each SNP followed by direct sequencing.

Statistical Analysis

Genotype associations with toxicity events or responsive tumor were first analyzed using univariate logistic regression. Genotypes generating P<0.20 were further analyzed using multivariate logistic regression including patient age, gender, baseline ECOG status and starting dose as covariates. Progression-free survival and overall survival were estimated by means of Kaplan-Meier method, while the associations of genotypes and patient characteristics with PFS and OS were analyzed using two-tailed log rank test. The effects of genotypes on the progression-free survival and overall survival were then evaluated using multivariate Cox regression model by including genotypes and patient characteristics with P<0.05 in univariate analysis. Only those patients for whom sunitinib was the first line treatment for mRCC were included in progression-free survival and overall survival analyses. In all analyses, missing data were kept missing except for baseline ECOG status, which was replaced with the median value. As this was designed as a validation study of previously observed correlations rather than an exploratory evaluation, multiple testing correction was not done.

Results

Patient Characteristics and Genotype Frequencies

The demographic and baseline clinical characteristics of the 97 patients included in this study are listed in Table 4. The polymorphism frequencies of the 6 SNPs are listed in Table 1. Hardy-Weinberg equilibrium held for all the 6 SNPs (P>0.05). After verifying pairwise linkage disequilibrium for ABCB1 3435C/T, ABCB1 1236C/T and ABCB1 2677G/TA by Chi-square test (P<0.05 in each pair) and phasing with PLINK, haplotype TTT was the most common haplotype and was found in 51 patients, among whom 8 were homozygous carriers. A complete list of haplotypes and their frequencies is provided in Table 5.

TABLE 4
Patient demographics and baseline characteristics
(n = 97)

| | | |
|---|---|---|
| Characteristic | No. | % |

| | |
|---|---|
| Median age when initiating | 58 |
| sunitinib, years | |
| Range | 18-79 |
| Gender | |

| | | |
|---|---|---|
| Male | 75 | 77.3 |
| Female | 22 | 22.7 |
| Ethnicity | | |
| Chinese | 86 | 88.7 |
| Malay | 7 | 7.2 |
| Indian | 4 | 4.1 |
| Baseline ECOG performance status | | |
| 0 | 27 | 27.8 |
| 1 | 51 | 52.6 |
| 2 | 13 | 13.4 |
| 3 | 6 | 6.2 |

| | |
|---|---|
| Median body surface area, m² | 1.63 |
| Range | 1.18-1.92 |
| Line of therapy | |

| | | |
|---|---|---|
| First line | 81 | 83.5 |
| Second or third line | 16 | 16.5 |
| Starting sunitinib dose, mg daily | | |
| <37.5 | 6 | 6.2 |
| 37.5 | 79 | 81.4 |
| 50 | 12 | 12.4 |
| Baseline chemistry and hematology | | |
| (n < 97 because of missing data) | | |

| | |
|---|---|
| Median aspartate transaminase, | 23 |
| U/L (n = 93) | |
| Range | 11-70 |
| Median alanine transaminase, U/L | 20 |
| (n = 93) | |
| Range | 8-135 |
| Median creatinine, μM (n = 96) | 104 |
| Range | 33-649 |
| Median hemoglobin, g/dL (n = 96) | 11.5 |
| Range | 5.9-15.8 |
| Median leukocyte, K/μL (n = 96) | 7.3 |
| Range | 1.4-24.1 |
| Median thrombocyte, K/μL (n = 96) | 280 |
| Range | 117-799 |

TABLE 5
ABCB1 haplotype frequencies estimated with and
without assuming associations (n = 92)

| 3435 | 1236 | 2677 | Without | |
|---|---|---|---|---|
| C/T | T/C | G/TA | association | With association |
| C | C | A | 0.028 | 0.096 |
| C | C | G | 0.114 | 0.222 |
| C | C | T | 0.083 | 0.000 |
| C | T | A | 0.049 | 0.029 |
| C | T | G | 0.199 | 0.214 |
| C | T | T | 0.146 | 0.059 |
| T | C | A | 0.017 | 0.000 |
| T | C | G | 0.070 | 0.037 |
| T | C | T | 0.051 | 0.008 |
| T | T | A | 0.030 | 0.000 |
| T | T | G | 0.122 | 0.032 |
| T | T | T | 0.089 | 0.303 |

Correlation of Genotypes to Toxicities

Univariate and multivariate logistic regression analyses for associations between genetic markers and clinical outcomes are listed in Table 6. Of note, the FLT3 738 TT genotype was associated with an 8.0-fold increase of the risk of leucopenia (P=0.03) and a 2.7-fold increase of the risk of neutropenia (P=0.04). The ABCB1 1236 T allele was correlated to a 3-fold decrease of the risk of neutropenia (P=0.03), a 25-fold decrease of the risk of diarrhea (P=0.0005) and a 10-fold decrease of the risk of early toxicity-necessitated treatment termination (P=0.04). The ABCB1 3435 T allele was associated with a 10-fold decrease of the risk of neutropenia (P=0.01) and a 3-fold decrease of the risk of diarrhea (P=0.02). The ABCB1 2677 T allele was correlated to a 3-fold decrease of the risk of neutropenia (P=0.04). The ABCB1 3435, 1236, 2677 TTT haplotype was correlated to a 10-fold decrease of the risk of neutropenia (P=0.03). The ABCG2 421 A allele was associated with a 3-fold decrease of the risk of neutropenia (P=0.03). No genotypes were correlated with thrombocytopenia or hand foot syndrome. The VEGFR2 1191C/T genotype was not associated with the toxicity endpoints.

TABLE 6
Factors associated with toxicities of sunitinib.
Only those with multivariate P ≦ 0.05 are listed.

| | | Univariate | | Multivariate^{a} | |
|---|---|---|---|---|---|
| | | OR (95% | | OR (95% | |
| Group | Prevalence^{b} | CI) | P | CI) | P |

Leucopenia (n = 85)

| | | | | | | |
|---|---|---|---|---|---|---|
| Age | | 11/85 | 1.0 (0.9, | 0.44 | | |
| | | | 1.0) | | | |
| Gender | Male vs. | 6/65 | 1 | | | |
| | Female | 5/20 | 3.3 (0.9, | 0.08 | | |
| | | | 12.4) | | | |
| Baseline | 0 | 1/25 | 1 | | | |
| ECOG | 1 | 9/44 | 6.2 (1.1, | 0.09 | | |
| | | | 117.6) | | | |
| | 2 | 1/12 | 2.2 (0.1, | 0.59 | | |
| | | | 58.7) | | | |
| | 3 | 0/4 | NR | 0.99 | | |
| Starting | <37.5 | 0/4 | 1 | | | |
| dose | 37.5 | 10/70 | NR | 0.99 | | |
| (mg) | 50 | 1/11 | NR | 0.99 | | |
| FLT3 738 | CC + CT | 2/42 | 1 | | 1 | |
| T/C | TT | 8/41 | 4.9 (1.1, | 0.06 | 8.0 (1.3, | 0.03 |
| | | | 33.6) | | 51.0) | |

Neutropenia (n = 88)

| | | | | | | |
|---|---|---|---|---|---|---|
| Age | | 40/88 | 1.0 (1.0, | 0.24 | | |
| | | | 1.1) | | | |
| Gender | Male | 27/68 | 1 | | | |
| | Female | 13/20 | 2.8 (1.0, | 0.05 | | |
| | | | 8.4) | | | |
| Baseline | 0 | 13/25 | 1 | | | |
| ECOG | 1 | 22/46 | 0.9 (0.3, | 0.74 | | |
| | | | 2.3) | | | |
| | 2 | 5/12 | 0.7 (0.2, | 0.56 | | |
| | | | 2.6) | | | |
| | 3 | 0/5 | NR | 0.99 | | |
| Starting | <37.5 | 1/5 | 1 | | | |
| dose | 37.5 | 32/72 | 3.2 (0.5, | 0.31 | | |
| (mg) | | | 64.3) | | | |
| | 50 | 7/11 | 7.0 (0.7, | 0.13 | | |
| | | | 165.7) | | | |
| FLT3 738 | CC + CT | 15/45 | 1 | | 1 | |
| T/C | TT | 23/41 | 2.6 (1.1, | 0.04 | 2.7 (1.1, | 0.04 |
| | | | 6.2) | | 7.2) | |
| ABCG2 | CC + AC | 23/42 | 1 | | 1 | |
| 421 C/A | AA | 16/44 | 0.5 (0.2, | 0.09 | 0.3 (0.1, | 0.03 |
| | | | 1.1) | | 0.9) | |
| ABCB1 | CC + CT | 27/52 | 1 | | 1 | |
| 1236 T/C | TT | 11/32 | 0.5 (0.2, | 0.12 | 0.3 (0.1, | 0.03 |
| | | | 1.2) | | 0.9) | |
| ABCB1 | Other | 20/35 | 1 | | 1 | |
| 2677 G/TA | TT + AT + GT | 20/53 | 0.5 (0.2, | 0.08 | 0.4 (0.1, | 0.04 |
| | | | 1.1) | | 0.9) | |
| ABCB1 | CC + CT. | 38/75 | 1 | | 1 | |
| 3435 C/T | TT | 1/12 | 0.1 (0.0, | 0.02 | 0.1 (0.0, | 0.01 |
| | | | 0.5) | | 0.4) | |
| ABCB1 | Other | 38/79 | 1 | | 1 | |
| haplotype^{c} | TTT/TTT | 1/8 | 0.2 (0.0, | 0.09 | 0.1 (0.0, | 0.03 |
| | | | 0.9) | | 0.5) | |

Diarrhea (n = 95)

| | | | | | | |
|---|---|---|---|---|---|---|
| Age | | 20/95 | 1.0 (1.0, | 0.62 | | |
| | | | 1.0) | | | |
| Gender | Male | 15/74 | 1 | | | |
| | Female | 5/21 | 1.2 (0.4, | 0.73 | | |
| | | | 3.7) | | | |
| Baseline | 0 | 6/27 | 1 | | | |
| ECOG | 1 | 9/50 | 0.8 (0.2, | 0.66 | | |
| | | | 2.6) | | | |
| | 2 | 4/12 | 1.8 (0.4, | 0.47 | | |
| | | | 7.9) | | | |
| | 3 | 1/6 | 0.7 (0.0, | 0.76 | | |
| | | | 5.6) | | | |
| Starting | <37.5 | 2/5 | 1 | | | |
| dose | 37.5 | 15/78 | 0.4 (0.1, | 0.28 | | |
| (mg) | | | 2.9) | | | |
| | 50 | 3/12 | 0.5 (0.1, | 0.54 | | |
| | | | 5.2) | | | |
| ABCB1 | CC | 11/34 | 1 | | 1 | |
| 3435 T/C | TT + CT | 9/60 | 0.4 (0.1, | 0.05 | 0.3 (0.1, | 0.02 |
| | | | 1.0) | | 0.8) | |
| ABCB1 | CC | 7/9 | 1 | | 1 | |
| 1236 T/C | TT + CT | 13/82 | 0.1 (0.0, | 0.0006 | 0.04 (0.0, | 0.0005 |
| | | | 0.3) | | 0.2) | |

Toxicity-necessitated treatment termination before completing the
first two cycles (n = 87)

| | | | | | | |
|---|---|---|---|---|---|---|
| Age | | 13/87 | 1.1 (1.0, | 0.01 | | |
| | | | 1.2) | | | |
| Gender | Male | 10/66 | 0.9 (0.2, | 0.92 | | |
| | Female | 3/21 | 3.5) | | | |
| Baseline | 0 | 2/26 | 1 | | | |
| ECOG | 1 | 7/44 | 2.3 (0.5, | 0.33 | | |
| | | | 16.1) | | | |
| | 2 | 2/12 | 2.4 (0.3, | 0.41 | | |
| | | | 22.4) | | | |
| | 3 | 2/5 | 8.0 (0.8, | 0.08 | | |
| | | | 93.2) | | | |
| Starting | <37.5 | 3/4 | 1 | | | |
| dose | 37.5 | 8/71 | 0.0 (0.0, | 0.01 | | |
| (mg) | | | 0.4) | | | |
| | 50 | 2/12 | 0.1 (0.0, | 0.05 | | |
| | | | 0.8) | | | |
| ABCB1 | CC + CT | 10/53 | 1 | | 1 | |
| 1236 T/C | TT | 2/30 | 0.3 (0.0, | 0.15 | 0.1 (0.0, | 0.04 |
| | | | 1.3) | | 0.7) | |

Abbreviations:
OR, ratio of the odds that the event occurs;
CI, confidence interval;
NR, not reached.
^{a}Including age, gender, starting dose and baseline ECOG status as covariates.
^{b}Number of cases affected by toxicity/total number of cases in the group.
^{c}ABCB1 3435C/T, 1236C/T, 2677G/TA haplotype.

Correlation of Genotypes to Tumor Response and Patient Survival

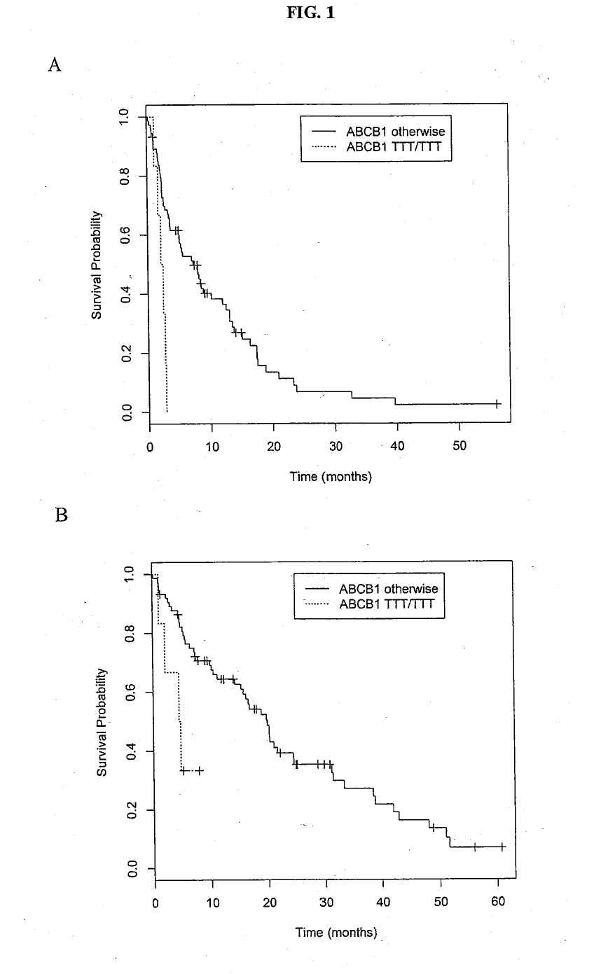

Primary sunitinib resistance, defined as progressive disease as the best response observed, was more common in carriers of the ABCB1 3435 TT genotype (P=0.02), ABCB1 2677 TT genotype (P=0.01) and the ABCB1 3435, 1236, 2677 TTT haplotype (P=0.004) (Table 7). Median progression-free survival of the 81 patients who received sunitinib as the first-line therapy was 8.1 months and median overall survival was 19.5 months. As in Table 8, after including starting dose as covariate based on univariate P<0.05, the ABCB1 3435, 1236, 2677 TTT haplotype was correlated with inferior progression-free survival (P=0.001) and overall survival (P=0.005). Survival curves depicting the correlations are provided in FIG. 1.

TABLE 7
Factors associated with the clinical benefit of
sunitinib (best response being partial response or stable disease)
(n = 90). Only those with multivariate P ≦ 0.05 are listed.

| | | | Univariate | | Multivariate^{a} | |
|---|---|---|---|---|---|---|
| | | Pre- | OR (95% | | OR (95% | |
| Group | | valence^{b} | CI) | P | CI) | P |
| Age | | 59/90 | 1.0 (0.9, | 0.38 | | |
| | | | 1.0) | | | |
| Gender | Male | 46/71 | 1 | | | |
| | Female | 13/19 | 1.2 (0.4, | 0.77 | | |
| | | | 3.7) | | | |
| Baseline | 0 | 20/25 | 1 | | | |
| ECOG | 1 | 31/49 | 0.4 (0.1, | 0.15 | | |
| | | | 1.3) | | | |
| | 2 | 5/11 | 0.2 (0.0, | 0.05 | | |
| | | | 0.9) | | | |
| | 3 | 3/5 | 0.4 (0.1, | 0.35 | | |
| | | | 3.4) | | | |
| Starting | ≦37.5 | 1/4 | 1 | | | |
| dose (mg) | 37.5 | 49/75 | 5.7 (0.7, | 0.14 | | |
| | | | 117.5) | | | |
| | 50 | 9/11 | 13.5 (1.1, | 0.06 | | |
| | | | 378.2) | | | |
| ABCB1 | Other | 55/79 | 1 | | 1 | |
| 2677 | TT | 4/11 | 0.3 (0.1, | 0.04 | 0.1 (0.0, | 0.01 |
| G/TA | | | 0.9) | | 0.6) | |
| ABCB1 | CC + CT. | 54/78 | 1 | | 1 | |
| 3435 | TT | 5/12 | 0.3 (0.1, | 0.07 | 0.2 (0.0, | 0.02 |
| C/T | | | 1.1) | | 0.7) | |
| ABCB1 | Other | 57/82 | 1 | | 1 | |
| haplotype^{c} | TTT/TTT | 2/8 | 0.2 (0.0, | 0.02 | 0.1 (0.0, | 0.004 |
| | | | 0.7) | | 0.3) | |

Abbreviations:
OR, ratio of the odds that the event occurs;
CI, confidence interval;
NR, not reached;
PR, partial response;
SD, stable disease.
^{a}Including age, gender, starting dose and baseline ECOG status as covariates.
^{b}Number of cases with PR or SD as the best response observed/total number of cases in the group.
^{c}ABCB1 3435C/T, 1236C/T, 2677G/TA haplotype.

TABLE 8
Survival analyses in mRCC patients receiving
sunitinib as first-line treatment (n = 81). Only those with
multivariate P ≦ 0.05 are listed.

| | | | Univariate | | Multivariate^{a} | |
|---|---|---|---|---|---|---|
| | | Median | HR (95% | | HR (95% | |
| Factor | No. | (months) | CI) | P | CI) | P |

Progression-free survival

| | | | | | | | |
|---|---|---|---|---|---|---|---|
| Age | | 81 | 8.1 | 1.0 (1.0, | 0.56 | | |
| | | | | 1.0) | | | |
| Gender | Female | 20 | 10.0 | 1 | 0.69 | | |
| | Male | 61 | 8.1 | 1.1 (0.6, | | | |
| | | | | 2.1) | | | |
| Baseline | 0 | 21 | 16.1 | 1 | 0.08 | | |
| ECOG | 1 | 43 | 6.9 | 2.2 (1.1, | | | |
| | | | | 4.4) | | | |
| | 2 | 11 | 3.3 | 2.8 (1.1, | | | |
| | | | | 7.0) | | | |
| | 3 | 6 | 12.9 | 2.8 (0.8, | | | |
| | | | | 10.3) | | | |
| Starting | ≦37.5 | 5 | 1.8 | 1 | 0.01 | | |
| dose | 37.5 | 71 | 8.3 | 0.2 (0.1, | | | |
| (mg) | | | | 0.8) | | | |
| | 50 | 5 | 17.3 | 0.1 (0.0, | | | |
| | | | | 0.6) | | | |
| MSKCC | Good | 7 | 12.6 | 1 | 0.14 | | |
| | Intermediate | 33 | 10.0 | 1.3 (0.5, | | | |
| | | | | 3.6) | | | |
| | Poor | 23 | 5.5 | 2.3 (0.8, | | | |
| | | | | 6.4) | | | |
| ABCB1 | CC + CT | 51 | 11.7 | 1 | 0.09 | 1 | 0.09 |
| 1236 | TT | 28 | 3.6 | 1.7 (0.9, | | 1.7 (0.9, | |
| T/C | | | | 3.0) | | 3.2) | |
| ABCB1 | Other | 71 | 8.4 | 1 | 0.09 | 1 | 0.44 |
| 2677 | TT | 10 | 2.7 | 2.3 (0.9, | | 1.5 (0.5, | |
| G/TA | | | | 6.0) | | 4.7) | |
| ABCB1 | CC + CT. | 70 | 8.4 | 1 | 0.19 | 1 | 0.19 |
| 3435 | TT | 10 | 2.7 | 1.7 (0.8, | | 1.7 (0.8, | |
| C/T | | | | 3.9) | | 3.9) | |
| Haplotype^{b} | Other | 74 | 8.4 | 1 | 0.0006 | 1 | 0.001 |
| | TTT/TTT | 6 | 2.4 | 4.9 (1.8, | | 5.5 (2.0, | |
| | | | | 13.6) | | 15.4) | |

Overall survival

| | | | | | | | |
|---|---|---|---|---|---|---|---|
| Age | | 81 | 19.5 | 1.0 (1.0, | 0.56 | | |
| | | | | 1.0) | | | |
| Gender | Female | 20 | 19.9 | 1 | 0.87 | | |
| | Male | 61 | 16.3 | 1.1 (0.6, | | | |
| | | | | 2.0) | | | |
| Baseline | 0 | 21 | 32.9 | 1 | 0.06 | | |
| ECOG | 1 | 43 | 19.6 | 1.9 (0.9, | | | |
| | | | | 3.9) | | | |
| | 2 | 11 | 5.7 | 3.0 (1.3, | | | |
| | | | | 7.1) | | | |
| | 3 | 6 | 15.7 | 2.7 (0.9, | | | |
| | | | | 8.0) | | | |
| Starting | ≦37.5 | 5 | 4.6 | 1 | <0.0001 | | |
| dose | 37.5 | 71 | 19.5 | 0.2 (0.1, | | | |
| (mg) | | | | 0.4) | | | |
| | 50 | 5 | 47.4 | 0.1 (0.0, | | | |
| | | | | 0.3) | | | |
| MSKCC | Good | 7 | 41.4 | 1 | 0.10 | | |
| | Intermediate | 33 | 19.6 | 2.2 (0.8, | | | |
| | | | | 6.5) | | | |
| | Poor | 23 | 14 | 3.1 (1.0, | | | |
| | | | | 9.1) | | | |
| ABCB1 | CC + CT | 51 | 20 | 1 | 0.09 | 1 | 0.07 |
| 1236 | TT | 28 | 10.4 | 1.7 (0.9, | | 1.7 (1.0, | |
| T/C | | | | 2.9) | | 3.1) | |
| ABCB1 | Other | 71 | 19.6 | 1 | 0.01 | 1 | 0.12 |
| 2677 | TT | 10 | 5.9 | 2.9 (1.3, | | 2.0 (0.8, | |
| G/TA | | | | 6.7) | | 5.0) | |
| ABCB1 | CC + CT. | 70 | 19.5 | 1 | 0.25 | 1 | 0.21 |
| 3435 | TT | 10 | 7.2 | 1.6 (0.7, | | 1.7 (0.7, | |
| C/T | | | | 3.6) | | 3.8) | |
| Haplotype^{b} | Other | 74 | 19.6 | 1 | 0.008 | 1 | 0.005 |
| | TTT/TTT | 6 | 4.6 | 3.9 (1.3, | | 5.0 (1.6, | |
| | | | | 11.7) | | 15.2) | |

Abbreviations:
HR, hazard ratio;
CI, confidence interval.
^{a}Including starting dose as covariate.
^{b}ABCB1 3435C/T, 1236C/T, 2677G/TA haplotype.

1. A method for determining the sensitivity of a patient with cancer to receptor tyrosine kinase inhibitor therapy, comprising:
a) isolating a nucleic acid sample from a biological sample obtained from the said patient;
b) screening said nucleic acid sample to determine the identity of at least one single nucleotide polymorphism (SNP) genotype selected from the group consisting of rs1933437, rs1045642, rs1128503, rs2032582 and rs2231142 and any combination thereof; and
c) determining the sensitivity of a patient with renal cancer to receptor tyrosine kinase inhibitor therapy based on the identity of at least one SNP genotype selected from the group consisting of rs1933437, rs1045642, rs1128503, rs2032582 and rs2231142 and any combination thereof,
wherein said sensitivity is selected from one or more of neutropenia, diarrhea, tumor response, early toxicity-necessitated treatment termination, overall survival and progression-free survival.

2. The method according to claim 1, wherein the cancer is renal cancer, neuroendocrine cancer, breast cancer, prostate cancer, cervical cancer, ovarian cancer, gastric cancer, colorectal cancer, pancreatic cancer, liver cancer, brain cancer, lung cancer, hematological malignancies, melanoma and sarcomas.

3. The method according to claim 2, wherein the renal cancer is metastatic renal cell carcinoma (mRCC); or wherein the patient suffers from mRCC; or wherein the patient suffers from mRCC and undergoes receptor tyrosine kinase inhibitor treatment.

4. (canceled)

5. The method according to claim 1, wherein the biological sample is obtained from the group consisting of frozen tissue, tissue biopsies, circulating cells, bodily fluids and cheek swab.

6. The method according to claim 5, wherein the biological sample is non-neoplastic or neoplastic.

7. (canceled)

8. The method according to claim 4, wherein the bodily fluids are selected from the group consisting of blood, saliva, ascites, effusions and urine.

9. The method according to claim 1, wherein the receptor tyrosine kinase inhibitor is a multikinase inhibitor for receptor tyrosine kinase including but not limited to sunitinib, pazopanib, axitinib, sorafenib, regorafenib, tivozantinib or combinations thereof.

10. The method according to claim 1, wherein the patient is a mammal or a human; or wherein the human is selected from the group consisting of an ethnic Asian, an ethnic Caucasian, and an ethnic African.

11. (canceled)

12. The method according to claim 1, for determining the risk of diarrhea during receptor tyrosine kinase inhibitor treatment, the method further comprising: (a) screening for at least one SNP genotype selected from the group consisting of rs1128503 and rs1045642, and (b) determining the risk of diarrhea based on the screening result obtained in operation (a).

13. The method according to claim 12, wherein in operation (b), with respect to rs1128503, the risk of diarrhea during receptor tyrosine kinase inhibitor treatment is higher for a CC genotype than a CT or TT genotype.

14. The method according to claim 12, wherein in operation (b), with respect to rs1045642, the risk of diarrhea during receptor tyrosine kinase inhibitor treatment is higher for a CC genotype than a CT or TT genotype.

15. The method according to claim 1, for determining the risk of neutropenia during receptor tyrosine kinase inhibitor treatment, the method further comprising: (a) screening for at least one SNP genotype selected from the group consisting of rs1933437, rs2032582, rs1045642, rs1128503 and rs2231142, and (b) determining the risk of neutropenia based on the screening result obtained in operation (a).

16. The method according to claim 15, wherein in operation (b), with respect to rs1933437, the risk of neutropenia during receptor tyrosine kinase inhibitor treatment is higher for a TT genotype than a CC or CT genotype.

17. The method according to claim 15, wherein in operation (b), with respect to rs2032582, the risk of neutropenia during receptor tyrosine kinase inhibitor treatment is higher for a AA or AG or GG genotype than a AT or GT or TT genotype.

18. The method according to claim 15, wherein in operation (b), with respect to rs1045642, the risk of neutropenia during receptor tyrosine kinase inhibitor treatment is higher for a CC or CT genotype than a TT genotype.

19. The method according to claim 15, wherein in operation (b), with respect to rs1128503, the risk of neutropenia during receptor tyrosine kinase inhibitor treatment is higher for a CC or CT genotype than a TT genotype.

20. The method according to claim 15, wherein in operation (b), with respect to rs2231142, the risk of neutropenia during receptor tyrosine kinase inhibitor treatment is higher for a CC or AC genotype than a AA genotype.

21. The method according to claim 15, wherein in operation (b), with respect to the three SNPs including rs1045642, rs2032582 and rs1128503, the risk of neutropenia during receptor tyrosine kinase inhibitor treatment is higher for allele combinations other than T allele homozygosity at all of the three SNP sites than for a TTT/TTT genotype.

22. The method according to claim 1, for determining the risk of early toxicity-necessitated treatment termination during receptor tyrosine kinase inhibitor treatment, the method comprising: (a) screening for the SNP genotype of rs1128503, and (b) determining the risk of early toxicity-necessitated treatment termination based on the genotyping result obtained in operation (a).

23. The method according to claim 22, wherein in operation (b), with respect to rs1128503, the risk of early toxicity-necessitated treatment termination during receptor tyrosine kinase inhibitor treatment is higher for a CC or CT genotype than a TT genotype.

24. The method according to claim 1, for determining the risk of primary tumour resistance to receptor tyrosine kinase inhibitor treatment, the method comprising: (a) screening for at least one SNP genotype selected from the group consisting of rs1045642, rs2032582 and rs1128503, and (b) determining the risk of primary tumour resistance to receptor tyrosine kinase inhibitor treatment based on the genotyping result obtained in operation (a).

25. The method according to claim 24, wherein in operation (b), with respect to rs1045642, the risk of primary tumour resistance to receptor tyrosine kinase inhibitor treatment is higher for a TT genotype than a CC or CT genotype.

26. The method according to claim 24, wherein in operation (b), with respect to rs2032582, the risk of primary tumour resistance to receptor tyrosine kinase inhibitor treatment is higher for a TT genotype than a AA or AG or GG or AT or GT genotype.

27. The method according to claim 24, wherein in operation (b), with respect to the three SNPs including rs1045642, rs2032582 and rs1128503, the risk of primary tumour resistance to receptor tyrosine kinase inhibitor therapy is higher for a TTT/TTT genotype than for allele combinations other than T allele homozygosity at all of the three SNP sites.

28. The method according to claim 1, for determining overall survival, during receptor tyrosine kinase inhibitor treatment, the method comprising: (a) screening for at least one SNP genotype selected from the group consisting of rs1045642, rs2032582 and rs1128503, and (b) determining overall survival, based on the genotyping result obtained in operation (a).

29. The method according to claim 28, wherein in operation (b), with respect to the three SNPs including rs1045642, rs2032582 and rs1128503, overall survival during receptor tyrosine kinase inhibitor treatment is lower for a TTT/TTT genotype than for allele combinations other than T allele homozygosity at all of the three SNPs.

30. The method according to claim 1, for determining progression-free survival, during receptor tyrosine kinase inhibitor treatment, the method comprising: (a) screening for at least one SNP genotype selected from the group consisting of rs1045642, rs2032582 and rs1128503, and (b) determining progression-free survival, based on the genotyping result obtained in operation (a).

31. The method according to claim 30, wherein in operation (b), with respect to the three SNP genotypes rs1045642, rs2032582 and rs1128503, progression-free survival during receptor tyrosine kinase inhibitor treatment is lower for a TTT/TTT genotype than for allele combinations other than T allele homozygosity at all of the three SNPs.

32. (canceled)

33. A kit for use according to a method for determining the sensitivity of a patient with cancer to receptor tyrosine kinase inhibitor therapy, comprising:
a) isolating a nucleic acid sample from a biological sample obtained from the said patient;
b) screening said nucleic acid sample to determine the identity of at least one single nucleotide polymorphism (SNP) genotype selected from the group consisting of rs1933437, rs1045642, rs1128503, rs2032582 and rs2231142 and any combination thereof; and
c) determining the sensitivity of a patient with renal cancer to receptor tyrosine kinase inhibitor therapy based on the identity of at least one SNP genotype selected from the group consisting of rs1933437, rs1045642, rs1128503, rs2032582 and rs2231142 and any combination thereof,
wherein said sensitivity is selected from one or more of neutropenia, diarrhea, tumor response, early toxicity-necessitated treatment termination, overall survival and progression-free survival, comprising components for the screening of said nucleic acid sample to determine the identity of at least one SNP genotype selected from the group consisting of rs1933437, rs1045642, rs1128503, rs2032582 and rs2231142 and any combination thereof.
